(19) United States
(12) Patent Application Publication
(10) Pub. No.: US 2018/0198163 A1
(43) Pub. Date: Jul. 12, 2018

(54) Non-aqueous electrolytes for lithium-ion batteries comprising an isocyanide

(71) Applicant: Gotion Inc., Fremont, CA (US)

(72) Inventors: Masaki Sekine, Tokyo (JP); Hiroyoshi Noguchi, Hyogo (JP); Martin Schulz-Dobrick, Fukushima (JP); Toshiyuki Edamoto, Hyogo (JP); Frederick Francois Chesneau, St. Leon-Rot (DE)

(21) Appl. No.: 15/741,003

(22) PCT Filed: Jun. 27, 2016
(86) PCT No.: PCT/EP2016/064780

§ 371 Date: Dec. 29, 2017

(30) Foreign Application Priority Data
Jul. 1, 2015 (EP) 15174748.2

(51) Int. Cl. H01M 10/0567; H01M 10/0525; H01M 10/0569; H01M 10/42; C07C 265/04; C07F 9/535; C07C 265/10; C07C 265/12; H01M 4/505; H01M 4/525; H01M 4/58; H01M 4/587

(52) U.S. Cl. CPC H01M 10/0567; H01M 10/0525; H01M 10/0569; H01M 10/4235; C07C 265/04; C07F 9/5355; H01M 2300/0037; C07C 265/12; H01M 4/505; H01M 4/525; H01M 4/5825; H01M 4/587; C07C 265/10

(57) ABSTRACT

A nonaqueous electrolyte composition containing at least one organic isocyanide of formula (I) R—NC, wherein:
R is selected from R¹, (CH₂)ₙL, and NP(R¹)₃;
L is selected from carboxylic ester groups, S-containing groups, N-containing groups, and P-containing groups which are substituted by one, two or three R¹;
R¹ is selected independently from C₁-C₁₀ alkyl, C₃-C₁₀ (hetero)cycloalkyl, C₂-C₁₀ alkenyl, C₃-C₇ (hetero)cycloalkenyl, C₂-C₁₀ alkynyl, C₅-C₇ (hetero)aryl, and C₆-C₁₃ (hetero)aralkyl,
   and n is an integer from 1 to 10;
   with proviso that C₃-C₁₀ (hetero)cycloalkyl is not morpholinyl.

The present invention relates to an electrolyte composition containing at least one organic isocyanide, to the use of organic isocyanides as additives in electrolyte compositions for electrochemical cells and to electrochemical cells comprising such electrolyte compositions.

Storing electrical energy is a subject of still growing interest. Efficient storage of electric energy would allow electric energy to be generated when it is advantageous and used when needed.

Secondary electrochemical cells are well suited for this purpose due to their reversible conversion of chemical energy into electrical energy and vice versa (rechargeability). Secondary lithium batteries are of special interest for energy storage since they provide high energy density and specific energy due to the small atomic weight of the lithium ion, and the high cell voltages that can be obtained (typically 3 to 5 V) in comparison with other battery systems. For that reason, these systems have become widely used as a power source for many portable electronics such as cellular phones, laptop computers, mini-cameras, etc.

In secondary lithium batteries like lithium ion batteries non-aqueous solvents like organic carbonates, ethers, esters and ionic liquids are used. Most state of the art lithium ion batteries in general comprise not a single solvent but a solvent mixture of different organic aprotic solvents. The contamination of the solvents by trace amounts of water from the solvents themselves or from other components such as electrodes of the lithium ion batteries is practically inevitable. An electrolyte composition usually contains at least one conducting salt dissolved in the solvent(s). The main electrolyte salt in current state electrolyte compositions for lithium-ion batteries is LiPF₆. LiPF₆ is very susceptible to the reaction with water and even trace amounts of water lead to the generation of hydrogen fluoride. The presence of water and hydrogen fluoride in the electrolyte composition have a negative effect on the battery. They can cause corrosion of electrodes, decomposition of other components present in the electrolyte composition, and/or generation of gasses resulting in a shortening of the battery life. It is known to reduce the water content of an electrolyte composition by adding a water-scavenging additive. It is on the other hand known that the formation of a solid electrolyte interface film may protect electrodes.

US 2013/0273427 A1 describes an electrochemical cell comprising a moisture scavenger, which may be added to the electrolyte composition or to other cell components like the cathode. The moisture scavenger may be an isocyanate like ethyl isocyanate or a silane compound like silazane.

JP 2011-028860 A discloses an electrochemical cell comprising an electrolyte composition containing isocyanates and di-isocyanates and an aromatic compound to scavenge water stemming from the cathode used in the electrochemical cell.

According to U.S. Pat. No. 6,077,628 carbodiimides are used to reduce the water content of the electrolyte solution for batteries and thereby preventing the reaction of LiPF₆ with water.

It is also known from JP 2001-313073 A to use carbodiimide as water scavenger in electrolyte compositions containing fluorinated conducting salts like LiPF₆ and LiBF₄ to prevent the generation of HF.

US 2015/0140395 describes electrolyte compositions for rechargeable lithium batteries containing a substituted morpholino compound as additive forming a solid electrolyte interface protection film on a surface of the negative electrode. The substituent may contain inter alia a functional group selected from —CN, —NC, —NCS and —SCN.

Despite the additives already known for scavenging water in electrolyte compositions for electrochemical cells, there is still the demand for further water scavenging additives, additives preventing the generation of HF from F-containing conducting salts for use in lithium batteries, and additives which will form more stable protective films on the electrodes. Another problem is the use of electrochemical cells at elevated or high temperatures. Usually battery fading occurs faster at temperatures above room temperature than at room temperature. Electrochemical cells having better high temperature charge-discharge cycle performance for use at higher temperatures are desired, too.

It is the object of the present invention to provide additives capable of scavenging water and of reducing the amount of HF in electrolyte compositions comprising F-containing conducting salts and to provide electrochemical cells exhibiting improved electrochemical performance at high temperatures.

This object is achieved by a nonaqueous electrolyte composition containing at least one organic isocyanide, preferably an organic isocyanide of formula (I)

R—N≡C (I)

wherein

R is selected from R¹, (CH₂)ₙL, and NP(OR¹)₃;

L is selected from carboxylic ester groups, S-containing groups, N-containing groups, and P-containing groups which are substituted by one, two or three R¹;

R¹ is selected independently from C₁-C₁₀ alkyl, C₃-C₁₀ (hetero)cycloalkyl, C₂-C₁₀ alkenyl, C₃-C₇ (hetero)cycloalkenyl, C₂-C₁₀ alkynyl, C₅-C₇ (hetero)aryl, and C₆-C₁₃ (hetero)aralkyl, wherein alkyl, (hetero)cycloalkyl, alkenyl, (hetero)cycloalkenyl, alkynyl, (hetero)aryl, and (hetero)aralkyl may be substituted by one or more substituents selected from F; NC; CN; C₁-C₆ alkyl optionally substituted by one or more substituents selected from F and CN; C₃-C₁₀ (hetero)cycloalkyl optionally substituted by one or more substituents selected from F and CN; C₂-C₆ alkenyl optionally substituted by one or more substituents selected from F and CN; C₅-C₇ (hetero)aryl optionally substituted by one or more substituents selected from F and CN; and C₆-C₁₃ (hetero)aralkyl optionally substituted by one or more substituents selected from F and CN; and wherein one or more CH₂ groups of alkyl, alkenyl, and alkynyl may be replaced by O or NH;

and n is an integer from 1 to 10;

with the proviso that C₃-C₁₀ (hetero)cycloalkyl is not morpholinyl.

This object is also accomplished by the use of organic isocyanides as additives in electrolyte compositions for electrochemical cells, in particular as water scavenging additive in electrolyte compositions for electrochemical cells, and by electrochemical cells comprising the electrolyte compositions.

Organic isocyanides exhibit superior water-scavenging reactivity compared to the conventional ones such as isocyanates or carbodiimides. Due to the higher water-scavenging ability of the isocyanide additive, the claimed non-aqueous electrolyte compositions exhibit low concentration of water and simultaneously the generation of hydrogen fluoride is effectively suppressed in case of a F-containing conducting salt present in the composition. Electrochemical cells comprising an electrolyte composition containing an organic isocyanide show improved electrochemical characteristics at high temperature.

In the following the invention is described in detail.

One aspect of the invention relates to electrolyte compositions containing at least one organic isocyanide. Organic isocyanides according to the present invention are compounds based on hydrocarbons carrying at least one isocyanide group. The hydrocarbons may contain one or more heteroatoms like oxygen, sulfur, nitrogen and phosphorus. Preferred organic isocyanides are organic isocyanides of formula (I)

R—N≡C (I)

wherein

R is selected from R¹, (CH₂)ₙL, and NP(OR¹)₃;

L is selected from carboxylic ester groups, S-containing groups, N-containing groups, and P-containing groups which are substituted by one, two or three R¹;

R¹ is selected independently from C₁-C₁₀ alkyl, C₃-C₁₀ (hetero)cycloalkyl, C₂-C₁₀ alkenyl, C₃-C₇ (hetero)cycloalkenyl, C₂-C₁₀ alkynyl, C₅-C₇ (hetero)aryl, and C₆-C₁₃ (hetero)aralkyl, wherein alkyl, (hetero)cycloalkyl, alkenyl, (hetero)cycloalkenyl, alkynyl, (hetero)aryl, and (hetero)aralkyl may be substituted by one or more substituents selected from F; NC; CN; C₁-C₆ alkyl optionally substituted by one or more substituents selected from F and CN; C₃-C₁₀ (hetero)cycloalkyl optionally substituted by one or more substituents selected from F and CN; C₂-C₆ alkenyl optionally substituted by one or more substituents selected from F and CN; C₅-C₇ (hetero)aryl optionally substituted by one or more substituents selected from F and CN; and C₆-C₁₃ (hetero)aralkyl optionally substituted by one or more substituents selected from F and CN; and

wherein one or more CH₂ groups of alkyl, alkenyl, and alkynyl may be replaced by O or NH;

and n is an integer from 1 to 10:

with the proviso that C₃-C₁₀ (hetero)cycloalkyl is not morpholinyl.

The term “C₁ to C₁₀ alkyl” as used herein means a straight or branched saturated hydrocarbon group with 1 to 10 carbon atoms having one free valence and includes, e.g., methyl, ethyl, n-propyl, isopropyl, n-butyl, isobutyl, tert-butyl, n-pentyl, iso-pentyl, 2-pentyl, 2,2-dimethylpropyl, n-hexyl, iso-hexyl, 2-ethyl hexyl, n-heptyl, iso-heptyl, n-octyl, iso-octyl, 1,1,3,3-tetramethylbutyl, n-nonyl, n-decyl and the like. Preferred are C₁-C₈ alkyl groups, more preferred are C₃-C₈ alkyl groups, and most preferred are iso-propyl, n-butyl, tert-butyl, n-pentyl, and 1,1,3,3-tetramethylbutyl.

The term “C₃ to C₁₀ (hetero)cycloalkyl” as used herein means a saturated 3- to 10-membered hydrocarbon cycle or polycycle having one free valence wherein one or more of the C— atoms of the saturated cycle may be replaced independently from each other by a heteroatom selected from N, S, O and P. Examples of C₃-C₁₀ (hetero)cycloalkyl are cyclopropyl, oxiranyl, cyclopentyl, pyrrolidyl, cyclohexyl, piperidyl, cycloheptyl, 1-adamantyl, and 2-adamantyl. Preferred are C₆-C₁₀ (hetero)cycloalkyl groups, in particular preferred are cyclohexyl, and 1-adamantyl. Also preferred are C₃ to C₁₀ cycloalkyl groups like cyclopropyl and cyclohexyl, in particular C₆ to C₁₀ cycloalkyl.

The term “C₂ to C₁₀ alkenyl” as used herein refers to an unsaturated straight or branched hydrocarbon group with 2 to 10 carbon atoms having one free valence. Unsaturated means that the alkenyl group contains at least one C—C double bond. C₂-C₁₀ alkenyl includes for example ethenyl, 1-propenyl, 2-propenyl, 1-n-butenyl, 2-n-butenyl, iso-butenyl, 1-pentenyl, 1-hexenyl, 1-heptenyl, 1-octenyl, 1-nonenyl, 1-decenyl and the like. Preferred are C₂-C₈ alkenyl groups, more preferred are C₂-C₆ alkenyl groups, even more preferred are C₂-C₄ alkenyl groups and in particular ethenyl and 1-propen-3-yl (allyl).

The term “C₃ to C₇ (hetero)cycloalkenyl” as used herein refers to an unsaturated 3- to 7-membered hydrocarbon cycle having one free valence and containing at least one C—C double bond wherein one or more of the C— atoms of the saturated cycle may be replaced independently from each other by a heteroatom selected from N, S, O and P. C₃-C₇ (hetero)cycloalkenyl includes for example cyclopentene and cyclohexene. Preferred are C₃-C₆ (hetero)cycloalkenyl.

The term “C₂ to C₁₀ alkynyl” as used herein refers to an unsaturated straight or branched hydrocarbon group with 2 to 10 carbon atoms having one free valence, wherein the hydrocarbon group contains at least one C—C triple bond. C₂-C₁₀ alkynyl includes for example ethynyl, 1-propynyl, 2-propynyl, 1-n-butynyl, 2-n-butynyl, 1-pentynyl, 1-hexynyl, 1-heptynyl, 1-octynyl, 1-nonynyl, 1-decynyl and the like. Preferred are C₂-C₈ alkynyl, more preferred are C₂-C₆ alkynyl, even more preferred are C₂-C₄ alkynyl, in particular preferred are ethynyl and 1-propyn-3-yl (propargyl).

The term “C₅ to C₇ (hetero)aryl” as used herein denotes an aromatic 5- to 7-membered hydrocarbon cycle having one free valence wherein one or more of the C— atoms of the aromatic cycle may be replaced independently from each other by a heteroatom selected from N, S, O and P. Examples of C₅-C₇ (hetero)aryl are furanyl, pyrrolyl, pyrazolyl, thienyl, pyridinyl, imidazolyl, and phenyl. Preferred is phenyl.

The term “C₆ to C₁₃ (hetero)aralkyl” as used herein denotes an aromatic 5- to 7-membered aromatic hydrocarbon cycle substituted by one or more C₁-C₆ alkyl, wherein one or more of the C— atoms of the aromatic cycle may be replaced independently from each other by a heteroatom selected from N, S, O and P, and one or more CH₂ groups of alkyl may be replaced by O or NH. The C₆-C₁₃ (hetero)aralkyl group contains in total 6 to 13 C-atoms and has one free valence. The free valence may be located at the (hetero)aromatic cycle or at a C₁-C₆ alkyl group, i.e. C₆-C₁₃ (hetero)aralkyl group may be bound via the aromatic part or via the alkyl part of the (hetero)aralkyl group. Examples of C₆-C₁₃ (hetero)aralkyl are methylphenyl, 2-methylfuranyl, 3-ethylpyridinyl 1,2-dimethylphenyl, 1,3-dimethylphenyl, 1,4-dimethylphenyl, ethylphenyl, 2-propylphenyl, and the like.

L is selected from carboxylic ester groups, S-containing groups, N-containing groups, and P-containing groups which are substituted by one, two or three R¹.

Examples of L are C(O)OR¹, OC(O)R¹, S(O)₂R¹, OS(O)₂R¹, S(O)₂OR¹, OS(O)₂OR¹, S(O)R¹, SR¹, P(O)(OR¹)₂, P(O)(OR¹)R¹, P(O)(R¹)₂, NP(R¹)₃, NP(OR¹)₃, NPR¹(OR¹)₂, and NP(R¹)₂OR¹, preferably L is selected from C(O)OR¹, OC(O)R¹, S(O)₂R¹, P(O)(OR¹)₂, (CH₂)ₙNP(R¹)₃, and NP(R¹)₃, more preferred L is selected from C(O)OR¹, S(O)₂R¹, P(O)(OR¹)₂, and NP(R¹)₃.

According to one embodiment L is C(O)OR¹ or OC(O)R¹.

R is preferably selected from R¹, (CH₂)ₙS(O)₂R¹, (CH₂)ₙP(O)(OR¹)₂, (CH₂)ₙNP(R¹)₃, NP(R¹)₃, and (CH₂)ₙC(O)OR¹.

Preferably R¹ is selected from C₁-C₁₀ alkyl, C₃-C₆ (hetero)cycloalkyl, C₅-C₇ (hetero)aryl, and C₆-C₁₃ (hetero)aralkyl, wherein alkyl, (hetero)cycloalkyl, (hetero)aryl, and (hetero)aralkyl may be substituted by one or more substituents selected from F; NC; CN; and C₁-C₆ alkyl which may be substituted by one or more substituents selected from F and CN; and wherein one or more CH₂ groups of alkyl may be replaced by O or NH with the proviso that C₃-C₁₀ (hetero)cycloalkyl is not morpholinyl.

n is preferably an integer selected from 1 to 6 and more preferred n is selected from 1 to 4.

Preferred compounds are compounds of formula (I) wherein R is selected from R¹, (CH₂)ₙS(O)₂R¹, (CH₂)ₙP(O)(OR¹)₂, (CH₂)ₙNP(R¹)₃, NP(R¹)₃, and (CH₂)ₙC(O)OR¹; R¹ is selected from C₁-C₁₀ alkyl, C₃-C₁₀ (hetero)cycloalkyl, C₅-C₇ (hetero)aryl, and C₆-C₁₃ (hetero)aralkyl, wherein alkyl, cycloalkyl, (hetero)aryl and (hetero)aralkyl may be substituted by one or more substituents selected from NC and C₁-C₆ alkyl and wherein one or more CH₂ groups of alkyl may be replaced by O or NH; and

n is an integer from 1 to 10;

with the proviso that C₃-C₁₀ (hetero)cycloalkyl is not morpholinyl.

Even more preferred compounds are compounds of formula (I) wherein R is selected from R¹, (CH₂)ₙS(O)₂R¹, (CH₂)ₙP(O)(OR¹)₂, (CH₂)ₙNP(R¹)₃, NP(R¹)₃, and (CH₂)ₙC(O)OR¹; R¹ is selected from C₁-C₁₀ alkyl, C₃-C₁₀ cycloalkyl, C₅-C₇ (hetero)aryl, and C₆-C₁₃ (hetero)aralkyl, wherein alkyl, cycloalkyl, (hetero)aryl and (hetero)aralkyl may be substituted by one or more substituents selected from NC and C₁-C₆ alkyl; and

n is an integer from 1 to 10.

Examples of organic isocyanides are tert-butyl isocyanide, 1-n-pentyl isocyanide, 1,1,3,3-tetramethylbutyl isocyanide, 1-adamantyl isocyanide, 2,6-dimethylphenyl isocyanide, 1,4-phenylene diisocyanide, p-toluenesulfonylmethyl isocyanide, diethyl isocyanomethylphosphate, (isocyanoimino)triphenylphosphorane, and ethyl isocyanoacetate.

Organic isocyanides are to some extent commercially available. The preparation of organic isocyanides is generally known to the person skilled in the art and is e.g. described in the following report: T. Matsuo, et al., J. Am. Chem. Soc. 2009, 131, 15124-15125.

The total concentration of the organic isocyanide(s) in the electrolyte composition is usually in the range of 0.01 to 5 wt.-%, based on the total weight of the electrolyte composition, preferably in the range of 0.025 to 3 wt.-%, and more preferred in the range of 0.05 to 2 wt.-%, based on the total weight of the electrolyte composition.

According to another aspect of the invention the organic isocyanides, as described above or as described as being preferred, are used as additives in electrolyte compositions for electrochemical cells, preferably the organic isocyanides are used as water scavenging additives and/or additives for improving the high temperature performance in electrolyte compositions for electrochemical cells. A water scavenging additive is an additive which reduces the amount of water present in a battery cell. This usually takes place by reaction or complexation of the water molecule by the water scavenging additive. It is preferred to use the organic isocyanides as additives in non-aqueous electrolyte compositions for electrochemical cells, more preferred the organic isocyanides are used as additives in non-aqueous electrolyte compositions for lithium batteries, even more preferred in non-aqueous electrolyte compositions for lithium ion batteries.

Accordingly, when an organic isocyanide is used as additive in an electrolyte composition, the concentration of the organic isocyanide(s) in the electrolyte composition is typically 0.01 to 5 wt.-%, preferred 0.025 to 3 wt.-% and most preferred 0.05 to 2 wt.-%, based on the total weight of the electrolyte composition. Usually the organic isocyanides are added to the electrolyte composition in the desired amount during or after manufacture of the electrolyte composition.

The electrolyte composition preferably contains at least one aprotic organic solvent, more preferred at least two aprotic organic solvents. According to one embodiment the electrolyte composition may contain up to ten aprotic organic solvents.

The at least one aprotic organic solvent is preferably selected from cyclic and acyclic organic carbonates, di-C₁-C₁₀-alkylethers, di-C₁-C₄-alkyl-C₂-C₆-alkylene ethers and polyethers, cyclic ethers, cyclic and acyclic acetales and ketales, orthocarboxylic acids esters, cyclic and acyclic esters of carboxylic acids, cyclic and acyclic sulfones, and cyclic and acyclic nitriles and dinitriles.

More preferred the at least one aprotic organic solvent is selected from cyclic and acyclic carbonates, di-C₁-C₁₀-alkylethers, di-C₁-C₄-alkyl-C₂-C₆-alkylene ethers and polyethers, cyclic and acyclic acetales and ketales, and cyclic and acyclic esters of carboxylic acids, even more preferred the electrolyte composition contains at least one aprotic organic solvent selected from cyclic and acyclic carbonates, and most preferred the electrolyte composition contains at least two aprotic organic solvents selected from cyclic and acyclic carbonates, in particular preferred the electrolyte composition contains at least one aprotic solvent selected from cyclic carbonates and at least one aprotic organic solvent selected from acyclic carbonates.

The aprotic organic solvents may be partly halogenated, e.g. they may be partly fluorinated, partly chlorinated or partly brominated, and preferably they may be partly fluorinated. “Partly halogenated” means, that one or more H of the respective molecule is substituted by a halogen atom, e.g. by F, Cl or Br. Preference is given to the substitution by F. The at least one solvent may be selected from partly halogenated and non-halogenated aprotic organic solvents i.e. the electrolyte composition may contain a mixture of partly halogenated and non-halogenated aprotic organic solvents.

Examples of cyclic carbonates are ethylene carbonate (EC), propylene carbonate (PC) and butylene carbonate (BC), wherein one or more H in may be substituted by F and/or an C₁ to C₄ alkyl group, e.g. 4-methyl ethylene carbonate, monofluoroethylene carbonate (FEC), and cis- and trans-difluoroethylene carbonate. Preferred cyclic carbonates are ethylene carbonate, monofluoroethylene carbonate, and propylene carbonate, in particular ethylene carbonate.

Examples of acyclic carbonates are di-C₁-C₁₀-alkylcarbonates, wherein each alkyl group is selected independently from each other, preferred are di-C₁-C₄-alkylcarbonates. Examples are e.g. diethyl carbonate (DEC), ethyl methyl carbonate (EMC), dimethyl carbonate (DMC), and methylpropyl carbonate. Preferred acyclic carbonates are diethyl carbonate (DEC), ethyl methyl carbonate (EMC), and dimethyl carbonate (DMC).

In one embodiment of the invention the electrolyte composition contains mixtures of acyclic organic carbonates and cyclic organic carbonates at a ratio by weight of from 1:10 to 10:1, preferred of from 3:7 to 8:2.

According to the invention each alkyl group of the di-C₁-C₁₀-alkylethers is selected independently from the other. Examples of di-C₁-C₁₀-alkylethers are dimethylether, ethylmethylether, diethylether, methylpropylether, diisopropylether, and di-n-butylether.

Examples of di-C₁-C₄-alkyl-C₂-C₆-alkylene ethers are 1,2-dimethoxyethane, 1,2-diethoxyethane, diglyme (diethylene glycol dimethyl ether), triglyme (triethyleneglycol dimethyl ether), tetraglyme (tetraethyleneglycol dimethyl ether), and diethylenglycoldiethylether.

Examples of suitable polyethers are polyalkylene glycols, preferably poly-C₁-C₄-alkylene glycols and especially polyethylene glycols. Polyethylene glycols may comprise up to 20 mol % of one or more C₁-C₄-alkylene glycols in copolymerized form. Polyalkylene glycols are preferably dimethyl- or diethyl-end-capped polyalkylene glycols. The molecular weight M_{w} of suitable polyalkylene glycols and especially of suitable polyethylene glycols may be at least 400 g/mol. The molecular weight M_{w} of suitable polyalkylene glycols and especially of suitable polyethylene glycols may be up to 5 000 000 g/mol, preferably up to 2 000 000 g/mol.

Examples of cyclic ethers are 1,4-dioxane, tetrahydrofuran, and their derivatives like 2-methyl tetrahydrofuran.

Examples of acyclic acetals are 1,1-dimethoxymethane and 1,1-diethoxymethane. Examples of cyclic acetals are 1,3-dioxane, 1,3-dioxolane, and their derivatives such as methyl dioxolane.

Examples of acyclic orthocarboxylic acid esters are tri-C₁-C₄ alkoxy methane, in particular trimethoxymethane and triethoxymethane. Examples of suitable cyclic orthocarboxylic acid esters are 1,4-dimethyl-3,5,8-trioxabicyclo[2.2.2]octane and 4-ethyl-1-methyl-3,5,8-trioxabicyclo[2.2.2]octane.

Examples of acyclic esters of carboxylic acids are ethyl and methyl formiate, ethyl and methyl acetate, ethyl and methyl proprionate, and ethyl and methyl butanoate, and esters of dicarboxylic acids like 1,3-dimethyl propanedioate. An example of a cyclic ester of carboxylic acids (lactones) is y-butyrolactone.

Examples of cyclic and acyclic sulfones are ethyl methyl sulfone, dimethyl sulfone, and tetrahydrothiophene-S,S-dioxide (sulfolane).

Examples of cyclic and acyclic nitriles and dinitriles are adipodinitrile, acetonitrile, propionitrile, and butyronitrile.

Viewed chemically, an electrolyte composition is any composition which comprises free ions and as a result is electrically conductive. The most typical electrolyte composition is an ionic solution, although molten electrolyte compositions and solid electrolyte compositions are likewise possible. An electrolyte composition of the invention is therefore an electrically conductive medium, primarily due to the presence of at least one substance which is present in a dissolved and/or molten state, i.e., an electrical conductivity supported by movement of ionic species.

The inventive electrolyte composition therefore usually contains at least one conducting salt. The electrolyte composition functions as a medium that transfers ions participating in the electrochemical reaction taking place in an electrochemical cell. The conducting salt(s) are usually present in the electrolyte in the solvated or melted state. In liquid or gel electrolyte compositions the conducting salt is usually solvated in the aprotic organic solvent(s). Preferably the conducting salt is a lithium salt. More preferred the conducting salt is selected from the group consisting of
Li[F₆₋ₓP(C_{y}F_{2y+1})ₓ], wherein x is an integer in the range from 0 to 6 and y is an integer in the range from 1 to 20;
Li[B(R^{I})₄], Li[B(R^{II})₂(OR^{II}O)] and Li[B(OR^{II}O)₂] wherein each R^{I} is independently from each other selected from F, C₁, Br, I, C₁-C₄ alkyl, C₂-C₄ alkenyl, C₂-C₄ alkynyl, OC₁-C₄ alkyl, OC₂-C₄ alkenyl, and OC₂-C₄ alkynyl wherein alkyl, alkenyl, and alkynyl may be substituted by one or more OR^{III}, wherein R^{III} is selected from C₁-C₆ alkyl, C₂-C₆ alkenyl, and C₂-C₆ alkynyl, and
(OR^{II}O) is a bivalent group derived from a 1,2- or 1,3-diol, a 1,2- or 1,3-dicarboxlic acid or a 1,2- or 1,3-hydroxycarboxylic acid, wherein the bivalent group forms a 5- or 6-membered cycle via the both oxygen atoms with the central B-atom;
LiClO₄; LiAsF₆; LiCF₃SO₃; Li₂SiF₆; LiSbF₆; LiAlCl₄, Li(N(SO₂F)₂), lithium tetrafluoro (oxalato) phosphate; lithium oxalate; and
salts of the general formula Li[Z(CₙF₂ₙ₊₁SO₂)ₘ], where m and n are defined as follows:
m=1 when Z is selected from oxygen and sulfur,
m=2 when Z is selected from nitrogen and phosphorus,
m=3 when Z is selected from carbon and silicon, and
n is an integer in the range from 1 to 20.

Suited 1,2- and 1,3-diols from which the bivalent group (OR^{II}O) is derived may be aliphatic or aromatic and may be selected, e.g., from 1,2-dihydroxybenzene, propane-1,2-diol, butane-1,2-diol, propane-1,3-diol, butan-1,3-diol, cyclohexyl-trans-1,2-diol and naphthalene-2,3-diol which are optionally are substituted by one or more F and/or by at least one straight or branched non fluorinated, partly fluorinated or fully fluorinated C₁-C₄ alkyl group. An example for such 1,2- or 1,3-diole is 1,1,2,2-tetra(trifluoromethyl)-1,2-ethane diol.

“Fully fluorinated C₁-C₄ alkyl group” means, that all H-atoms of the alkyl group are substituted by F.

Suited 1,2- or 1,3-dicarboxlic acids from which the bivalent group (OR^{II}O) is derived may be aliphatic or aromatic, for example oxalic acid, malonic acid (propane-1,3-dicarboxylic acid), phthalic acid or isophthalic acid, preferred is oxalic acid. The 1,2- or 1,3-dicarboxlic acid are optionally substituted by one or more F and/or by at least one straight or branched non fluorinated, partly fluorinated or fully fluorinated C₁-C₄ alkyl group.

Suited 1,2- or 1,3-hydroxycarboxylic acids from which the bivalent group (OR^{II}O) is derived may be aliphatic or aromatic, for example salicylic acid, tetrahydro salicylic acid, malic acid, and 2-hydroxy acetic acid, which are optionally substituted by one or more F and/or by at least one straight or branched non fluorinated, partly fluorinated or fully fluorinated C₁-C₄ alkyl group. An example for such 1,2- or 1,3-hydroxycarboxylic acids is 2,2-bis(trifluoromethyl)-2-hydroxy-acetic acid.

Examples of Li[B(R^{I})₄], Li[B(R^{I})₂(OR^{II}O)] and Li[B(OR^{II}O)₂] are LiBF₄, lithium difluoro oxalato borate and lithium dioxalato borate.

Preferably the at least one conducting salt is selected from F-containing conducting lithium salts, more preferred from LiPF₆, LiBF₄, LiClO₄, LiN(SO₂C₂F₅)₂, LiN(SO₂CF₃)₂, LiN(SO₂F)₂, and LiPF₃(CF₂CF₃)₃, even more preferred the conducting salt is selected from LiPF₆, LiBF₄, and LiN(SO₂CF₃)₂, and the most preferred conducting salt is LiPF₆.

The at least one conducting salt is usually present at a minimum concentration of at least 0.1 mol/l, preferably the concentration of the at least one conducting salt is 0.5 to 2 mol/l based on the entire electrolyte composition.

The electrolyte composition according to the present invention may further contain at least one additive different from organic isocyanides. This additive may be selected from polymers, SEI forming additives, flame retardants, overcharge protection additives, wetting agents, additional HF and/or H₂O scavenger, stabilizer for LiPF₆ salt, ionic salvation enhancer, corrosion inhibitors, gelling agents, and the like.

Polymers may be added to electrolyte compositions containing a solvent or solvent mixture in order to convert liquid electrolytes into quasi-solid or solid electrolytes and thus to improve solvent retention, especially during ageing and to prevent leakage of solvent from the electrochemical cell. Examples for polymers used in electrolyte compositions are polyvinylidene fluoride, polyvinylidene-hexafluoropropylene copolymers, polyvinylidene-hexafluoropropylene-chlorotrifluoroethylene copolymers, Nafion, polyethylene oxide, polymethyl methacrylate, polyacrylonitrile, polypropylene, polystyrene, polybutadiene, polyethylene glycol, polyvinylpyrrolidone, polyaniline, polypyrrole and/or polythiophene.

Examples of flame retardants are organic phosphorus compounds like cyclophosphazenes, phosphoramides, alkyl and/or aryl tri-substituted phosphates, alkyl and/or aryl di- or tri-substituted phosphites, alkyl and/or aryl di-substituted phosphonates, alkyl and/or aryl tri-substituted phosphines, and fluorinated derivatives thereof.

Examples of HF and/or H₂O scavenger different from organic isocyanides are optionally halogenated cyclic and acyclic silylamines, carbodiimides and isocyanates.

Examples of overcharge protection additives are cyclohexylbenzene, o-terphenyl, p-terphenyl, and biphenyl and the like, preferred are cyclohexylbenzene and biphenyl.

SEI forming additives are known to the person skilled in the art. An SEI forming additive according to the present invention is a compound which decomposes on an electrode to form a passivation layer on the electrode which prevents degradation of the electrolyte composition and/or the electrode. In this way, the lifetime of a battery is significantly extended. Preferably the SEI forming additive forms a passivation layer on the anode. An anode in the context of the present invention is understood as the negative electrode of a battery. Preferably, the anode has a reduction potential of 1 Volt or less vs. Li⁺/Li redox couple, such as a graphite anode. In order to determine if a compound qualifies as anode film forming additive, an electrochemical cell can be prepared comprising a graphite electrode and a lithium-ion containing cathode, for example lithium cobalt oxide, and an electrolyte containing a small amount of said compound, typically from 0.01 to 10 wt.-% of the electrolyte composition, preferably from 0.05 to 5 wt.-% of the electrolyte composition. Examples of SEI forming additives are vinylene carbonate and its derivatives such as vinylene carbonate and methylvinylene carbonate; fluorinated ethylene carbonate and its derivatives such as monofluoroethylene carbonate, cis- and trans-difluorocarbonate; propane sultone and its derivatives; ethylene sulfite and its derivatives; oxalate comprising compounds such as lithium oxalate, oxalato borates including dimethyl oxalate, lithium bis(oxalate) borate, lithium difluoro (oxalato) borate, and ammonium bis(oxalato) borate, and oxalato phosphates including lithium tetrafluoro (oxalato) phosphate; and ionic compounds containing a compound of formula (II)

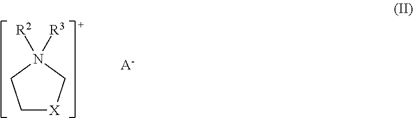

wherein

X is CH₂ or NR^{a},

R² is selected from C₁ to C₆ alkyl,

R³ is selected from —(CH₂)ᵤ—SO₃—(CH₂)ᵥ-R^{b},

—SO₃— is —O—S(O)₂— or —S(O)₂—O—, preferably —SO₃— is —O—S(O)₂—,

u is an integer from 1 to 8, preferably u is 2, 3 or 4, wherein one or more CH₂ groups of the —(CH₂)ᵤ— alkylene chain which are not directly bound to the N-atom and/or the SO₃ group may be replaced by O and wherein two adjacent CH₂ groups of the —(CH₂)ᵤ— alkylene chain may be replaced by a C—C double bond, preferably the —(CH₂)ᵤ— alkylene chain is not substituted and u u is an integer from 1 to 8, preferably u is 2, 3 or 4,

v is an integer from 1 to 4, preferably v is 0,

R^{a} is selected from C₁ to C₆ alkyl,

R^{b} is selected from C₁-C₂₀ alkyl, C₂-C₂₀ alkenyl, C₂-C₂₀ alkynyl, C₆-C₁₂ aryl, and C₆-C₂₄ aralkyl, which may contain one or more F, and wherein one or more CH₂ groups of alkyl, alkenyl, alkynyl and aralkyl which are not directly bound to the SO₃ group may be replaced by O, preferably R^{b} is selected from C₁-C₆ alkyl, C₂-C₄ alkenyl, and C₂-C₄ alkynyl, which may contain one or more F, and wherein one or more CH₂ groups of alkyl, alkenyl, alkynyl and aralkyl which are not directly bound to the SO₃ group may be replaced by O, preferred examples of R^{b} include methyl, ethyl, trifluoromethyl, pentafluoroethyl, n-propyl, n-butyl, n-hexyl, ethenyl, ethynyl, allyl or prop-1-yn-yl,

and an anion A⁻ selected from bisoxalato borate, difluoro (oxalato) borate, [F_{z}B(CₘF₂ₘ₊₁)_{4-z}]⁻, [F_{y}P(CₘF₂ₘ₊₁)_{6-y}]⁻, [CₘF₂ₘ₊₁)₂P(O)O]⁻, [CₘF₂ₘ₊₁P(O)O₂]²⁻, [O—C(O)—CₘF₂ₘ₊₁]⁻, [O—S(O)₂—CₘF₂ₘ₊₁]⁻, [N(C(O)—CₘF₂ₘ₊₁)₂]⁻, [N(S(O)₂—CₘF₂ₘ₊₁)₂]⁻, [N(C(O)—CₘF₂ₘ₊₁)(S(O)₂—CₘF₂ₘ₊₁)]⁻, [N(C(O)—CₘF₂ₘ₊₁)(C(O)F)]⁻, [N(S(O)₂—CₘF₂ₘ₊₁)(S(O)₂F)]⁻, [N(S(O)₂F)₂]⁻, [C(C(O)—CₘF₂ₘ₊₁)₃]⁻, [C(S(O)₂—CₘF₂ₘ₊₁)₃]⁻, wherein m is an integer from 1 to 8, z is an integer from 1 to 4, and y is an integer from 1 to 6,

Preferred anions A⁻ are bisoxalato borate, difluoro (oxalato) borate, [F₃B(CF₃)]⁻, [F₃B(C₂F₅)]⁻, [PF₆]⁻, [F₃P(C₂F₅)₃]⁻, [F₃P(C₃F₇)₃]⁻, [F₃P(C₄F₉)₃]⁻, [F₄P(C₂F₅)₂]⁻, [F₄P(C₃F₇)₂]⁻, [F₄P(C₄F₉)₂]⁻, [F₅P(C₂F₅)]⁻, [F₅P(C₃F₇)]⁻ or [F₅P(C₄F₉)]⁻, [(C₂F₅)₂P(O)O]⁻, [(C₃F₇)₂P(O)O]⁻ or [(C₄F₉)₂P(O)O]⁻. [C₂F₅P(O)O₂]2−, [C₃F₇P(O)O₂]2−, [C₄F₉P(O)O₂]²⁻, [O—C(O)CF₃]⁻, [O—C(O)C₂F₅]⁻, [O—C(O)C₄F₉]⁻, [O—S(O)₂CF₃]⁻, [O—S(O)₂C₂F₅]⁻, [N(C(O)C₂F₅)₂]⁻, [N(C(O)(CF₃)₂]⁻, [N(S(O)₂CF₃)₂]⁻, [N(S(O)₂C₂F₅)₂]⁻, [N(S(O)₂C₃F₇)₂]⁻, [N(S(O)₂CF₃) (S(O)₂C₂F₅)]⁻, [N(S(O)₂C₄F₉)₂]⁻, [N(C(O)CF₃)(S(O)₂CF₃)]⁻, [N(C(O)C₂F₅)(S(O)₂CF₃)]⁻ or [N(C(O)CF₃)(S(O)₂—C₄F₉)]⁻, [N(C(O)CF₃)(C(O)F)]⁻, [N(C(O)C₂F₅)(C(O)F)]⁻, [N(C(O)C₃F₇)(C(O)F)]⁻, [N(S(O)₂CF₃)(S(O)₂F)]⁻, [N(S(O)₂C₂F₅)(S(O)₂F)]⁻, [N(S(O)₂C₄F₉)(S(O)₂F)]⁻, [C(C(O)CF₃)₃]⁻, [C(C(O)C₂F₅)₃]⁻ or [C(C(O)C₃F₇)₃]⁻, [C(S(O)₂CF₃)₃]⁻, [C(S(O)₂C₂F₅)₃]⁻, and [C(S(O)₂C₄F₉)₃].

More preferred the anion A⁻ is selected from bisoxalato borate, difluoro (oxalato) borate, CF₃SO₃⁻, and [PF₃(C₂F₅)₃⁻].

Compounds of formula (II) are described in WO 2013/026854 A1.

Preferred SEI-forming additives are oxalato borates, fluorinated ethylene carbonate and its derivatives, vinylene carbonate and its derivatives, and compounds of formula (II). More preferred are lithium bis(oxalato) borate (LiBOB), vinylene carbonate, monofluoro ethylene carbonate, and compounds of formula (II), in particular monofluoro ethylene carbonate, and compounds of formula (II).

A compound added as additive may have more than one effect in the electrolyte composition and the device comprising the electrolyte composition. E.g. lithium oxalato borate may be added as additive enhancing the SEI formation but it may also be added as conducting salt.

According to a preferred embodiment of the present invention the electrolyte composition contains at least one SEI forming additive, all as described above or as described as being preferred.

In one embodiment of the present invention, the electrolyte composition contains:

(i) at least one organic aprotic solvent,

(ii) at least one conducting salt,

(iii) at least one organic isocyanide, and

(iv) optionally at least one additive different from organic isocyanides.

The electrolyte composition preferably contains components (i) to (iv) in the following concentrations ranges

(i) at least 70 wt.-% of at least one organic aprotic solvent;

(ii) 0.1 to 25 wt.-% of at least one conducting salt;

(iii) 0.01 to 5 wt.-% of at least one organic isocyanide; and

(iv) 0 to 25 wt.-% of at least one additive different from organic isocyanides; based on the total weight of the electrolyte composition.

The electrolyte composition is nonaqueous. This means the electrolyte composition contains only nonaqueous solvents. Nonaqueous solvents of technical grade may contain some water, usually only in traces. Therefore, the nonaqueous electrolyte composition contains some water introduced by the nonaqueous solvents used for the preparation of the electrolyte composition.

The water content of the inventive electrolyte composition is preferably below 100 ppm, based on the weight of the electrolyte composition, more preferred below 50 ppm, most preferred below 30 ppm. The water content may be determined by titration according to Karl Fischer, e.g. described in detail in DIN 51777 or ISO760: 1978.

The electrolyte composition contains preferably less than 50 ppm HF, based on the weight of the electrolyte composition, more preferred less than 40 ppm HF, most preferred less than 30 ppm HF. The HF content may be determined by titration according to potentiometric or potentiographic titration method or ion chromatography.

The present invention also provides a method for reducing the water content of a non-aqueous electrolyte composition without increasing the HF content by adding at least one organic isocyanide to the electrolyte composition.

The electrolyte compositions described herein may be prepared by methods known to the person skilled in the field of the production of electrolytes, generally by dissolving the conducting salt in the corresponding solvent mixture, adding the isocyanide(s) of the formula (I) according to the invention, and optionally additional additives, as described above.

A possible preparation process of the inventive electrolyte compositions comprises the steps
a) providing at least one organic aprotic solvent;
b) adding together or independently from each other the at least one organic isocyanide, at least one conducting salt, and optionally at least one additive different from organic isocyanides.

Although one of the main source of undesired traces of water in an electrochemical cell are typically the solvents used for the preparation of the electrolyte compositions, the organic isocyanides can also scavenge water stemming from other sources, e.g. water introduced by the conducting salt or by further additives present in the electrolyte composition. The isocyanides may also be effective in scavenging water originating from other components of the electrochemical cell, e.g. water introduced by the cathode or the anode.

The inventive electrolyte composition is preferably liquid at working conditions; more preferred it is liquid at 1 bar and 25° C., even more preferred the electrolyte composition is liquid at 1 bar and −15° C., in particular the electrolyte composition is liquid at 1 bar and −30° C., even more preferred the electrolyte composition is liquid at 1 bar and −50° C.

The electrolyte compositions are used in electrochemical cells like lithium batteries, double layer capacitors, and lithium ion capacitors, preferably the inventive electrolyte compositions are used in lithium batteries and more preferred in lithium ion batteries. The terms “electrochemical cell” and “battery” are used interchangeably herein.

The invention further provides an electrochemical cell comprising the electrolyte composition as described above or as described as being preferred. The electrochemical cell may be a lithium battery, a double layer capacitor, or a lithium ion capacitor

The general construction of such electrochemical devices is known and is familiar to the person skilled in this art—for batteries, for example, in Linden's Handbook of Batteries (ISBN 978-0-07-162421-3).

Preferably the electrochemical cell is a lithium battery. The term “lithium battery” as used herein means an electrochemical cell, wherein the anode comprises lithium metal or lithium ions sometime during the charge/discharge of the cell. The anode may comprise lithium metal or a lithium metal alloy, a material occluding and releasing lithium ions, or other lithium containing compounds; e.g. the lithium battery may be a lithium ion battery, a lithium/sulphur battery, or a lithium/selenium sulphur battery.

In particular preferred the electrochemical device is a lithium ion battery, i.e. a secondary lithium ion electrochemical cell comprising a cathode comprising a cathode active material that can reversibly occlude and release lithium ions and an anode comprising an anode active material that can reversibly occlude and release lithium ions. The terms “secondary lithium ion electrochemical cell” and “(secondary) lithium ion battery” are used interchangeably within the present invention.

The at least one cathode active material preferably comprises a material capable of occluding and releasing lithium ions selected from lithiated transition metal phosphates and lithium ion intercalating metal oxides.

Examples of lithiated transition metal phosphates are LiFePO₄ and LiCoPO₄, examples of lithium ion intercalating metal oxides are LiCoO₂, LiNiO₂, mixed transition metal oxides with layer structure having the general formula Li_{(1+z)}[NiₐCO_{b}Mn_{c}]_{(1-z)}O₂₊ₑ wherein z is 0 to 0.3; a, b and c may be same or different and are independently 0 to 0.8 wherein a+b+c=1; and −0.1≤e≤0.1, and manganese-containing spinels like LiMnO₄ and spinels of general formula Li₁₊ₜM₂₋ₜO_{4-d} wherein d is 0 to 0.4, t is 0 to 0.4 and M is Mn and at least one further metal selected from the group consisting of Co and Ni, and Li_{(1+g)}[NiₕCOᵢAlⱼ]_{(1-g)}O₂₊ₖ. Typical values for g, h, l, j and k are: g=0, h=0.8 to 0.85, i=0.15 to 0.20, j=0.02 to 0.03 and k=0.

The cathode may further comprise electrically conductive materials like electrically conductive carbon and usual components like binders. Compounds suited as electrically conductive materials and binders are known to the person skilled in the art. For example, the cathode may comprise carbon in a conductive polymorph, for example selected from graphite, carbon black, carbon nanotubes, graphene or mixtures of at least two of the aforementioned substances. In addition, the cathode may comprise one or more binders, for example one or more organic polymers like polyethylene, polyacrylonitrile, polybutadiene, polypropylene, polystyrene, polyacrylates, polyvinyl alcohol, polyisoprene and copolymers of at least two comonomers selected from ethylene, propylene, styrene, (meth)acrylonitrile and 1,3-butadiene, especially styrene-butadiene copolymers, and halogenated (co)polymers like polyvinlyidene chloride, polyvinyl chloride, polyvinyl fluoride, polyvinylidene fluoride (PVdF), polytetrafluoroethylene, copolymers of tetrafluoroethylene and hexafluoropropylene, copolymers of tetrafluoroethylene and vinylidene fluoride and polyacrylnitrile.

The anode comprised within the lithium batteries of the present invention comprises an anode active material that can reversibly occlude and release lithium ions or is capable to form an alloy with lithium. In particular carbonaceous material that can reversibly occlude and release lithium ions can be used as anode active material. Carbonaceous materials suited are crystalline carbon such as a graphite material, more particularly, natural graphite, graphitized cokes, graphitized MCMB, and graphitized MPCF; amorphous carbon such as coke, mesocarbon microbeads (MCMB) fired below 1500° C., and mesophase pitch-based carbon fiber (MPCF); hard carbon and carbonic anode active material (thermally decomposed carbon, coke, graphite) such as a carbon composite, combusted organic polymer, and carbon fiber.

Further anode active materials are lithium metal, or materials containing an element capable of forming an alloy with lithium. Non-limiting examples of materials containing an element capable of forming an alloy with lithium include a metal, a semimetal, or an alloy thereof. It should be understood that the term “alloy” as used herein refers to both alloys of two or more metals as well as alloys of one or more metals together with one or more semimetals. If an alloy has metallic properties as a whole, the alloy may contain a nonmetal element. In the texture of the alloy, a solid solution, an eutectic (eutectic mixture), an intermetallic compound or two or more thereof coexist. Examples of such metal or semimetal elements include, without being limited to, titanium (Ti), tin (Sn), lead (Pb), aluminum, indium (In), zinc (Zn), antimony (Sb), bismuth (Bi), gallium (Ga), germanium (Ge), arsenic (As), silver (Ag), hafnium (Hf), zirconium (Zr) yttrium (Y), and silicon (Si). Metal and semimetal elements of Group 4 or 14 in the long-form periodic table of the elements are preferable, and especially preferable are titanium, silicon and tin, in particular silicon. Examples of tin alloys include ones having, as a second constituent element other than tin, one or more elements selected from the group consisting of silicon, magnesium (Mg), nickel, copper, iron, cobalt, manganese, zinc, indium, silver, titanium (Ti), germanium, bismuth, antimony and chromium (Cr). Examples of silicon alloys include ones having, as a second constituent element other than silicon, one or more elements selected from the group consisting of tin, magnesium, nickel, copper, iron, cobalt, manganese, zinc, indium, silver, titanium, germanium, bismuth, antimony and chromium.

A further possible anode active material is silicon which is able to intercalate lithium ions. The silicon may be used in different forms, e.g. in the form of nanowires, nanotubes, nanoparticles, films, nanoporous silicon or silicon nanotubes. The silicon may be deposited on a current collector. The current collector may be a metal wire, a metal grid, a metal web, a metal sheet, a metal foil or a metal plate. Preferred the current collector is a metal foil, e.g. a copper foil. Thin films of silicon may be deposited on metal foils by any technique known to the person skilled in the art, e.g. by sputtering techniques. One possibility of preparing Si thin film electrodes are described in R. Elazari et al.; Electrochem. Comm. 2012, 14, 21-24. It is also possible to use a silicon/carbon composite as anode active material according to the present invention.

Other possible anode active materials are lithium ion intercalating oxides of Ti.

Preferably the anode active material is selected from carbonaceous material that can reversibly occlude and release lithium ions, particularly preferred the carbonaceous material that can reversibly occlude and release lithium ions is selected from crystalline carbon, hard carbon and amorphous carbon, in particular preferred is graphite. In another preferred embodiment the anode active is selected from silicon that can reversibly occlude and release lithium ions, preferably the anode comprises a thin film of silicon or a silicon/carbon composite. In a further preferred embodiment the anode active is selected from lithium ion intercalating oxides of Ti.

The anode and cathode may be made by preparing an electrode slurry composition by dispersing the electrode active material, a binder, optionally a conductive material and a thickener, if desired, in a solvent and coating the slurry composition onto a current collector. The current collector may be a metal wire, a metal grid, a metal web, a metal sheet, a metal foil or a metal plate. Preferred the current collector is a metal foil, e.g. a copper foil or aluminum foil.

The inventive lithium batteries may contain further constituents customary per se, for example separators, housings, cable connections etc. The housing may be of any shape, for example cuboidal or in the shape of a cylinder, the shape of a prism or the housing used is a metal-plastic composite film processed as a pouch. Suited separators are for example glass fiber separators and polymer-based separators like polyolefin separators.

Several inventive lithium batteries may be combined with one another, for example in series connection or in parallel connection. Series connection is preferred. The present invention further provides for the use of inventive lithium ion batteries as described above in devices, especially in mobile devices. Examples of mobile devices are vehicles, for example automobiles, bicycles, aircraft, or water vehicles such as boats or ships. Other examples of mobile devices are those which are portable, for example computers, especially laptops, telephones or electrical power tools, for example from the construction sector, especially drills, battery-driven screwdrivers or battery-driven tackers. But the inventive lithium ion batteries can also be used for stationary energy stores.

Even without further statements, it is assumed that a skilled person is able to utilize the above description in its widest extent. Consequently, the preferred embodiments and examples are to be interpreted merely as a descriptive enclosure which in no way has any limiting effect at all.

The invention is illustrated by the examples which follow, which do not, however, restrict the invention.

1. Evaluation of Water Scavenging and Suppression of HF Generation

1.1 Electrolyte Compositions

An electrolyte composition was prepared by mixing LiPF₆, ethylene carbonate (EC), and ethyl methyl carbonate (EMC) yielding a solution containing 12.7 wt.-% LiPF₆, 26.2 wt.-% EC and 61.1 wt.-% EMC. The water content of the solution was 20 ppm and the HF content was 30 ppm as determined by Karl-Fischer titration and ion chromatography, respectively. Different water scavengers selected from octadecyl isocyanate, dicyclohexyl carbodiimide, 1-n-pentyl isocyanide, ethyl isocyanoacetate, and (isocyanoimino)triphenylphosphorane were added at a concentration of 0.050 mol/kg. The different electrolyte compositions are displayed in Table 1.

TABLE 1

| Example | Water scavenger |
|---|---|
| Comparative | none |
| Example 1 | |
| Comparative | Octadecyl isocyanate |
| Example 2 | |
| Comparative | Dicyclohexyl carbodiimide |
| Example 3 | |
| Example 1 | 1-n-Pentyl isocyanide |
| Example 2 | Ethyl isocyanoacetate |
| Example 3 | (Isocyanoimino)triphenylphosphorane |

1.2 Water Scavenging and HF Formation

To each of the above-formulated solutions specific amounts of water were added to yield solutions with a water content of 250 ppm except for example 3. For Example 3, specific amounts of water were added to yield solutions with a water content of 500 ppm. Subsequently for each solution the concentration of water and that of hydrogen fluoride was measured periodically by Karl-Fischer titration and by ion chromatography, respectively. The results are shown below in Table 2.

TABLE 2
Concentration change of water and HF in the electrolyte compositions

| | | Concentration change of water/HF | | | |
|---|---|---|---|---|---|
| | | after the addition of water [ppm] | | | |
| | | After 3 h | | After 24 h | |
| | Additive | Water | HF | Water | HF |
| Comparative | none | 147 | 282 | 48 | 626 |
| Example 1 | | | | | |
| Comparative | Octadecyl isocyanate | 60 | 194 | 2 | 191 |
| Example 2 | | | | | |
| Comparative | Dicyclohexyl | 209 | 12 | 216 | 14 |
| Example 3 | carbodiimide | | | | |
| Example 1 | 1-n-Pentyl isocyanide | 57 | 35 | 58 | 37 |
| Example 2 | Ethyl isocyanoacetate | 85 | 58 | 89 | 54 |
| Example 3*¹ | (Isocyanoimino) | 466 | 28 | 209 | 30 |
| | triphenylphosphorane | | | | |

*¹500 ppm water was contained initially.

As shown in Table 2, in the sample without any water-scavenging additive, the concentration of water decreased gradually and simultaneously the concentration of HF significantly increased (Comparative Example 1). For the solution containing isocyanate as a water-scavenging additive, faster decrease of the concentration of water was observed, but still considerable increase of the concentration of HF occurred (Comparative Example 2). For the electrolyte composition containing carbodiimide, although the generation of HF was effectively suppressed, no water scavenging could be observed (Comparative Example 3). Only in the case of the electrolyte composition containing isocyanide, certain water-scavenging was observed and at the same time the generation of HF was effectively suppressed (Examples 1, 2 and 3).

2. Evaluation of Electrochemical Performance of the Electrolyte Compositions with Lithium Iron Phosphate as Cathode Active Material

2.1 Fabrication of the Cathode

90 wt.-% of lithium iron phosphate (LFP), 5 wt.-% of carbon black, and 5 wt.-% of polyvinylidene fluoride (pVdF) was added to N-methyl pyrrolidone (NMP) and stirred to form a smooth slurry. This slurry was coated onto aluminum foil (thickness=15 μm) by using a roll coater and dried under ambient temperature. This electrode tape was then kept at 130° C. under vacuum for 8 h to be ready for use. The thickness of the cathode active material was found to be 72 μm, which was corresponding to a loading amount of 14.4 mg/cm² and to a density of the active material of 2.0 g/cm².

2.2 Fabrication of the Anode

95.7 wt.-% of graphite, 0.5 wt.-% of carbon black, and 3.8 wt.-% of mixture of carboxymethyl-cellulose (CMC) and styrene-butadiene rubber (SBR) was added to deionized water and stirred to form a smooth slurry. This slurry was coated onto copper foil (thickness=10 μm) by using a roll coater and dried under ambient temperature. This electrode tape was then kept at 90° C. under vacuum for 8 h to be ready for use. The thickness of the anode active material was found to be 72 μm, which corresponds a loading amount of 7.1 mg/cm² and a density of the active material of 1.5 g/cm².

2.3 Formulation of Electrolyte Compositions

Comparative example 4 was prepared by mixing 12.5 wt.-% of LiPF₆, 25.6 wt.-% of EC, 59.9 wt.-% of EMC, and 2.0 wt.-% of vinylene carbonate (VC) to form a homogeneous solution. Comparative example 5 was prepared as described for comparative examples 4 wherein finally 250 ppm of water was added. Comparative example 6 was prepared as described for comparative examples 4 wherein finally 1000 ppm of water was added. Comparative examples 7 and 8 were prepared as described for comparative examples 4 wherein 0.050 mol/kg of octadecyl isocyanate or dicyclohexylcarbodiimide were also added and finally 250 ppm of water was added. Inventive example 4 was prepared as described for comparative examples 4 wherein 0.050 mol/kg of 1-n-pentyl isocyanide was also added. Inventive example 5 was prepared as described for comparative examples 4 wherein 0.050 mol/kg of ethyl isocyanoacetate was also added and finally 250 ppm of water was added.

2.4 Fabrication of the test cells

The cathode tape and the anode tape fabricated as described above were cut into pieces of cathode (50 mm×50 mm) and anode (52 mm×52 mm). For each cell one of these cathodes and one of these anodes were attached by sonication with aluminum current collector (thickness=15 μm), and then placed in an aluminum-laminated bag. A polyolefin separator (thickness=16 μm, porosity=31.0%) was placed in-between the cathode and the anode. Electrolyte compositions of comparative examples 4, 5, 6, 7, or 8 or inventive example 2, 3, 4, 5, or 6 was injected into this bag (300 μL) under inert atmosphere. The open-end of the bag was sealed by vacuum heat sealer. The nominal capacity of these pouch-type test cells was 52 mAh.

2.5 Electrochemical Performance of the Test Cells at High Temperature

Electrochemical cycle tests were carried out to see the fading of the discharge capacity of the test cells during charge-discharge cycling at 45° C. Voltage was controlled referring to the voltage between the cathode and the anode. For charging, Constant Current, Constant Voltage (CCCV) mode was employed; the current density was 1 C mA and the cut-off voltage was 3.7 V. When the current reached 0.02 mA or less, the charging stopped. After 5 min resting time, discharging started. For discharging, Constant Current (CC) mode was employed; the current density was 1 C mA, and the cut-off voltage was 2.0 V. The charge-discharge cycling was carried out in a constant temperature oven at 45° C. The results are summarized below in Table 3.

TABLE 3
Capacity retention during cycling at 45° C.

| | | | | | | | Discharge |
|---|---|---|---|---|---|---|---|
| | | | Discharge capacity | | | | capacity |
| | Water scavenger | Water | [mAh/g] | | | | retention |
| | (0.050 mol/kg) | content | 1st | 250th | 500th | 1000th | (1000th/1st) |
| Comparative | — | — | 135.8 | 124.7 | 120.3 | 113.3 | 83.4% |
| Example 4 | | | | | | | |
| Comparative | — | 250 ppm | 134.2 | 122.3 | 100.0 | 84.3 | 62.8% |
| Example 5 | | | | | | | |
| Comparative | — | 1000 ppm | 135.5 | 105.5 | 90.0 | 85.4 | 63.0% |
| Example 6 | | | | | | | |
| Comparative | Octadecyl isocyanate | 250 ppm | 133.6 | 121.4 | 117.0 | 110.7 | 82.9% |
| Example 7 | | | | | | | |
| Comparative | Dicyclohexyl carbodiimide | 250 ppm | 134.8 | 126.8 | 120.3 | 112.0 | 83.1% |
| Example 8 | | | | | | | |
| Example 4 | 1-n-Pentyl isocyanide | 250 ppm | 134.6 | 125.9 | 121.8 | 116.7 | 86.7% |
| Example 5 | Ethyl isocyano- | 250 ppm | 135.3 | 126.3 | 121.7 | 115.2 | 85.2% |
| | acetate | | | | | | |

The discharge capacity of the 1st cycle was used as basis for the calculation of the discharge capacity retention

First of all, it was confirmed that addition of isocyanide in the standard condition didn't cause any harmful effect on the high temperature cycle performance (Table 3, Examples 4 and 5 vs. Comparative Example 4). Then, the effect of the contamination of water into the cell was confirmed; the presence of a considerable amount of water in the electrolyte composition causes significant fading of the capacity after 500 cycles or even after only 250 cycles (Comparative Example 5 and 6). Addition of conventional water scavenger such as isocyanate and carbodiimide could improve the capacity retention, though the discharge capacities through the cycle life were at every moment slightly lower than the comparative example without additional water (Comparative Examples 7 and 8 compared to Comparative Example 4). In contrast, when an isocyanide was added as water scavenger, the capacity fading was effectively suppressed even after 1000 cycles and capacity retention was even higher than in the comparative example without additional water (Examples 4 and 5 compared to Comparative Example 4). Moreover, notably, the electrolyte compositions containing both 250 ppm water and one of the isocyanides exhibited the best cycle performance among all; even better than the cell using the comparative electrolyte composition which did not contain additional water. Thus, the isocyanide effectively improve the cycling performance of the cell at high temperature even in the presence of sub-stantial amount of contaminating water.

3. Evaluation of Electrochemical Performance of the Electrolyte Compositions with a Lithiated Mixed Oxide of Ni, Co and Mn as Cathode Active Material

3.1 Fabrication of the Cathode

93 wt.-% of LiNiₓCo_{y}Mn_{z}O₂(x:y:z=5:2:3), 1.5 wt.-% of carbon black, 1.5 wt.-% of graphite, and 4 wt.-% of polyvinylidene fluoride (pVdF) were added to N-methyl pyrrolidone (NMP) and stirred to form a smooth slurry. This slurry was coated onto aluminum foil (thickness=15 μm) by using a roll coater and dried under ambient temperature. This electrode tape was then roll-pressed and dried at 130° C. under vacuum for 8 h, to be ready for use. The thickness of the cathode active material was found to be 45 μm, which was corresponding to a loading amount of 12.2 mg/cm² and to a density of the active material of 2.9 g/cm².

Water content of this cathode tape was measured before use by using a moisture sensor: COM-PUTRAC Vapor Pro, Model CT3100, by Arizona Instrument. The cathode contained 200 ppm of water.

3.2 Fabrication of the Anode

95.7 wt.-% of graphite, 0.5 wt.-% of carbon black, and 3.8 wt.-% of mixture of carboxymethyl-cellulose (CMC) and styrene-butadiene rubber (SBR) were added to deionized water and stirred to form a smooth slurry. This slurry was coated onto copper foil (thickness=10 μm) by using a roll coater and dried under ambient temperature. This electrode tape was then roll-pressed and dried at 90° C. under vacuum for 8 h, to be ready for use. The thickness of the anode active material was found to be 47 μm, which corresponds a loading amount of 6.8 mg/cm² and a density of the active material of 1.5 g/cm².

3.3 Formulation of Electrolyte Compositions

LiPF₆ (12.7 wt.-%), EC (25.9 wt.-%), DEC (60.4 wt.-%) and an additive (1.00 wt.-%) chosen from ethyl isocyanoacetate, tert-butyl isocyanide, 1,1,3,3-tetramethylbutyl isocyanide, 1-adamantyl isocyanide, 2,6-dimethylphenyl isocyanide, 1,4-phenylene diisocyanide, p-toluenesulfonylmethyl isocyanide, diethyl isocyanomethylphosphonate, or (isocyanoimino)triphenylphosphorane were mixed to form homogeneous solutions for Examples 6 to 14. Comparative Example 9 was prepared as for Examples 6 to 14 without adding an additive.

3.4 Fabrication of the Test Cells

The cathode tape and the anode tape fabricated as described above were cut into pieces of cathode (50 mm×50 mm) and anode (52 mm×52 mm). For each cell, one piece of this cathode and one piece of this anode were attached by sonication with aluminum current collector (thickness=15 μm), and then placed in an aluminum-laminated bag. A polyolefin separator (thickness=16 μm, porosity=31.0%) was placed in-between the cathode and the anode. One of electrolyte compositions described above was injected into this bag (300 μL) under inert atmosphere. The open-end of the bag was sealed by vacuum heat sealer. The nominal capacity of these pouch-type test cells was 45 mAh.

3.5 Electrochemical Performance of the Test Cells at High Temperature

Electrochemical cycle tests were carried out to see the fading of the discharge capacity of the test cells during charge-discharge cycling at 60° C. Voltage was controlled referring to the voltage between the cathode and the anode. For charging, Constant Current-Constant Voltage (CCCV) mode was employed; the current density was 1 C mA and the cut-off voltage was 4.2 V.

When the current reached 0.02 mA or less, the charging stopped. After 5 min resting time, discharging started. For discharging, Constant Current (CC) mode was employed; the current density was 1 C mA, and the cut-off voltage was 2.7 V. The charge-discharge cycling was carried out in a constant temperature oven at 60° C. The results are summarized below in Table 4. By using an isocyanide as an additive, capacity retention during cycling was moderately to significantly improved.

TABLE 4
Capacity retention during 60° C. cycling

| | | Discharge capacity |
|---|---|---|
| | | retention |
| Example | Additive | 300^{th}/1^{st} |
| Comparative | none | 60% |
| Example 9 | | |
| Example 6 | Ethyl isocyanoacetate | 67% |
| Example 7 | tert-Butyl isocyanide | 74% |
| Example 8 | 1,1,3,3-Tetramethylbutyl isocyanide | 78% |
| Example 9 | 1-Adamantyl isocyanide | 79% |
| Example 10 | 2,6-Dimethylphenyl isocyanide | 67% |
| Example 11 | 1,4-Phenylene diisocyanide | 78% |
| Example 12 | p-Toluenesulfonylmethyl isocyanide | 68% |
| Example 13 | Diethyl isocyanomethylphosphonate | 70% |
| Example 14 | (Isocyanoimino)triphenylphosphorane | 68% |

The invention claimed is:

1. A nonaqueous electrolyte composition containing at least one organic isocyanide of formula (I)
R—N≡C (I)
wherein
R is selected from R¹, (CH₂)ₙL, and NP(R¹)₃;
L is selected from carboxylic ester groups, S-containing groups, N-containing groups, and P-containing groups which are substituted by one, two or three R¹;
R¹ is selected independently from C₁-C₁₀ alkyl, C₃-C₁₀ (hetero)cycloalkyl, C₂-C₁₀ alkenyl, C₃-C₇ (hetero)cycloalkenyl, C₂-C₁₀ alkynyl, C₅-C₇ (hetero)aryl, and C₆-C₁₃ (hetero)aralkyl, wherein alkyl, (hetero)cycloalkyl, alkenyl, (hetero)cycloalkenyl, alkynyl, (hetero)aryl, and (hetero)aralkyl may be substituted by one or more substituents selected from F; NC; CN; C₁-C₆ alkyl optionally substituted by one or more substituents selected from F and CN; C₃-C₁₀ (hetero)cycloalkyl optionally substituted by one or more substituents selected from F and CN; C₂-C₆ alkenyl optionally substituted by one or more substituents selected from F and CN; C₅-C₇ (hetero)aryl optionally substituted by one or more substituents selected from F and CN; and C₆-C₁₃ (hetero)aralkyl optionally substituted by one or more substituents selected from F and CN; and wherein one or more CH₂ groups of alkyl, alkenyl, and alkynyl may be replaced by O or NH;
and n is an integer from 1 to 10;
with proviso that C₃-C₁₀ (hetero)cycloalkyl is not morpholinyl.

2. The electrolyte composition according to claim 1, wherein R¹ is selected from C₁-C₁₀ alkyl, C₃-C₆ (hetero)cycloalkyl, C₅-C₇ (hetero)aryl, and C₆-C₁₃ (hetero)aralkyl, wherein alkyl, (hetero)cycloalkyl, (hetero)aryl, and (hetero)aralkyl are optionally substituted by one or more substituents selected from F; NC; CN; C₃-C₁₀ (hetero)cycloalkyl is optionally substituted by one or more substituents selected from F and CN; and C₁-C₆ alkyl is optionally substituted by one or more substituents selected from F and CN; and wherein one or more CH₂ groups of alkyl are optionally replaced by O or NH with proviso that C₃-C₁₀ (hetero)cycloalkyl is not morpholinyl.

3. The electrolyte composition according to claim 1, wherein L is selected from C(O)OR¹, OC(O)R¹, S(O)₂R¹, OS(O)₂R¹, S(O)₂OR¹, OS(O)₂OR¹, S(O)R¹, SR¹, P(O)(OR¹)₂, P(O)(OR¹)R¹, P(O)(R¹)₂, NP(R¹)₃, NP(OR¹)₃, NPR¹(OR¹)₂, and NP(R¹)₂OR¹.

4. The electrolyte composition according to claim 1, wherein
R is selected from R¹, (CH₂)ₙS(O)₂R¹, (CH₂)ₙP(O)(OR¹)₂, (CH₂)NP(R¹)₃, NP(R¹)₃, and (CH₂)ₙC(O)OR¹;
R¹ is selected from C₁-C₁₀ alkyl, C₃-C₁₀ (hetero)cycloalkyl, C₅-C₇ (hetero)aryl, and C₆-C₁₃ (hetero)aralkyl, wherein alkyl, cycloalkyl, (hetero)aryl and (hetero)aralkyl arem optionally substituted by one or more substituents selected from NC and C₁-C₆ alkyl and wherein one or more CH₂ groups of alkyl are optionally replaced by O or NH; and
n is an integer from 1 to 10;
with the proviso that C₃-C₁₀ (hetero)cycloalkyl is not morpholinyl.

5. The electrolyte composition according to claim 1, wherein the organic isocyanide is selected from tert-butyl isocyanide, 1-n-pentyl isocyanide, 1,1,3,3-tetramethylbutyl isocyanide, 1-adamantyl isocyanide, 2,6-dimethylphenyl isocyanide, 1,4-phenylene diisocyanide, p-toluenesulfonylmethyl isocyanide, diethyl isocyanomethylphosphate, (isocyanoimino)triphenylphosphorane, and ethyl isocyanoacetate.

6. The electrolyte composition according to claim 1, wherein the total concentration of organic isocyanides in the electrolyte composition is in the range of 0.01 to 5 wt.-% of the total weight of the electrolyte composition.

7. The electrolyte composition according to claim 1, wherein the electrolyte composition contains at least one aprotic organic solvent selected from cyclic and acyclic organic carbonates, di-C₁-C₁₀-alkylethers, di-C₁-C₄-alkyl-C₂-C₆-alkylene ethers and polyethers, cyclic ethers, cyclic and acyclic acetales and ketales, orthocarboxylic acids esters, cyclic and acyclic esters and diesters of carboxylic acids, cyclic and acyclic sulfones, and cyclic and acyclic nitriles and dinitriles.

8. The electrolyte composition according to claim 1, wherein the electrolyte composition contains at least one conducting salt selected from lithium salts.

9. The electrolyte composition according to claim 1, wherein the electrolyte composition contains at least one additive different from organic isocyanides.

10. The electrolyte composition according to claim 1, wherein the electrolyte composition contains
(i) at least 70 wt.-% of at least one organic aprotic solvent;
(ii) 0.1 to 25 wt.-% of at least one conducting salt;
(iii) 0.01 to 5 wt.-% of at least one organic isocyanide; and
(iv) 0 to 25 wt.-% of at least one additive different from organic isocyanides, based on the total weight of the electrolyte composition.

11. Use of organic isocyanides of formula (I) as defined in claim 1 as additives in a non-aqueous electrolyte composition for electrochemical cell.

12. The use according to claim 11, wherein the organic isocyanides of formula (I) are used as water scavenging additives.

13. An electrochemical cell comprising the electrolyte composition according to claim 1.

14. The electrochemical cell according to claim 13 wherein the electrochemical cell is a lithium battery.

15. The electrochemical cell according to claim 14 wherein the electrochemical cell comprises a cathode containing at least one cathode active material selected from lithium intercalating transition metal oxides and lithium transition metal phosphates.
